(19) United States
(12) Patent Application Publication
(10) Pub. No.: US 2016/0341708 A1
(43) Pub. Date: Nov. 24, 2016

(54) METHOD FOR DETERMINING SPOILAGE OF HIGH PROTEIN FOODS

(71) Applicant: HILL'S PET NUTRITION, INC., Topeka, KS (US)

(72) Inventor: Gerhard Zehentbauer, Lawrence, KS (US)

(73) Assignee: Hill's Pet Nutrition, Inc., Topeka, KS (US)

(21) Appl. No.: 15/106,612

(22) PCT Filed: Dec. 19, 2013
(86) PCT No.: PCT/US13/76446

§ 371 Date: Jun. 20, 2016

(51) Int. Cl. G01N 33/02; A23K 20/147; A23K 50/40

(52) U.S. Cl. CPC G01N 33/02; A23K 50/40; A23K 20/147

(57) ABSTRACT

The invention provides methods for determining the degree of spoilage of a high protein food by determining the level of particular acids, e.g., acetic acid, in the food.

### BACKGROUND

Food spoilage is a metabolic process that results in changes which are undesirable or unhealthy for the consumer. Food intended for consumption by both humans and animals, e.g., pets, can be prone to spoilage. In particular, foods with high protein content can be susceptible to food spoilage by the metabolism of the protein content of the food by microorganisms, including fungi and, in particular, bacteria. Deterioration or spoilage of food during storage not only results in a reduction of quality in the food product but has significant economic and health issues. In the area of health concerns, the proliferation of bacteria in protein based food during storage can lead to many forms of food-borne illness.

Bacteria are known to spoil food by producing high levels of biogenic amines. Biogenic amines are themselves toxic at higher levels, and are also indicators of food spoilage and microbial proliferation. Directly determining the amounts of biogenic amines in food is possible via conventional techniques such as reversed phase HPLC followed by UV or fluorescence detection; however, such techniques can be slow, expensive and cumbersome.

Food producers need to be able to reliably detect and quantify spoilage of ingredients to ensure quality and avoid costly and dangerous product contamination. For example, if the spoilage is not detected early and contaminates a large batch of product, the entire batch may ultimately need to be destroyed. It would be desirable to have a quick, efficient and cost-effective technique to determine the levels of biogenic amines in foods, e.g. high protein foods comprising meat or fish products. Such a technique could improve food quality, reduce incidences of food-borne illness, and improve the economics of food sales by reducing losses due to expiration and avoiding contamination of products with spoiled ingredients.

### BRIEF SUMMARY

We have discovered that biogenic amine levels in high protein foods, such as foods comprising meat or fish products, correlate closely with levels of particular acids, especially levels of acetic acid. This correlation is novel and unexpected. The invention thus provides, in one embodiment, a method for detecting and/or quantifying food spoilage in a high protein food, comprising:
(a) obtaining a sample of the high protein food,
(b) determining the amount of an acid in the sample, wherein the acid is selected from acetic, propanoic, butanoic, 2-methylbutanoic, 3-methylbutanoic, and 4-methylpentanoic acid, e.g. acetic acid, and wherein the level of acid correlates with the degree of spoilage.

This method provides significant advantages over direct measurement of biogenic amines, because there are already numerous commercially available, inexpensive, and highly sensitive techniques available for measuring specific acids, e.g., acetic acid.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

It has now been surprisingly discovered that the level of particular acids, e.g., acetic acid, in high protein foods is correlated with the levels of certain biogenic amities. Thus, acetic acid can be used as a chemical marker to track spoilage. While correlation between biogenic amine levels and lactic acid has been reported sporadically, evidently due to the involvement in some cases of lactic acid producing bacteria in spoilage, there have not been any reports of correlation of biogenic amine levels with acetic acid levels in high protein foods. Acetic acid is generally associated with microbial fermentation of sugars, either in an oxidative process with ethanol as an intermediate, or by anaerobic bacteria directly from sugars. It is not typically identified as a by-product of protein degradation.

Biogenic amines are formed during food spoilage by the decarboxylation of the amino acids comprising the food proteins. Although amino acid decarboxylases are not widely distributed among bacteria, bacteria known to have relevant enzymes include Bacillus sp., Citrobacter sp., Clostridium sp., Pseudomonas sp., Shigella sp., Escherichia coli, Lactobacillus sp., Streptococcus sp., Klebseilla sp., Morganella sp. e.g., Morganella morganii, Enterococcus sp., e.g., Enterococcus faecalis and Proteus sp., e.g., Proteus vulgaris.

Among the biogenic amine produced by bacteria during food spoilage, the predominant and most important are 1,4-diaminobutane (putrescine) and 1,5-diaminopentane (cadaverine). Putrescine is produced by the action of the bacterial enzyme omithine decarboxylase, which degrades the amino acid L-omithine to produce putrescine and carbon dioxide. Cadaverine is produced by the bacterial enzyme lysine decarboxylase, which breaks down the amino acid L-lysine to cadaverine and carbon dioxide.

It is known that the levels of putrescine and cadaverine are indicative of the degree of food spoilage. We have found that there is a strong correlation between the levels of putrescine and/or cadaverine and the levels of particular acids, especially acetic acid. Therefore, determining the level of such acids in a high protein food will provide an indicator of the degree of food spoilage.

As used herein “high protein food” is meant a food product or ingredient containing protein in an amount of from about 20 to 95% on a dry weight basis. For example, high protein ingredients as used in pet food may contain about 50-90% protein: whey protein is 80-90% protein by weight, high-quality fishmeal normally contains between 60% and 72% crude protein by weight, chicken meal (rendered and reduced to approximately 10% water) contains about 65% crude protein by weight. A finished dog food product would typically have about 18 35% crude protein on a dry weight basis, while a finished cat food would typically have about 26-45% crude protein on a dry weight basis. Spoilage of any of these products could be measured using the methods of the invention. In particular embodiments the high protein food products contain relatively low levels of fermentable carbohydrates, such as sugars, e.g., less than 5%, e.g., less than 2%.

The high protein food used in the method or assay of the invention can be food for humans or any animal, preferably a mammal, more preferably a companion animal. The term “companion animal” refers to any animal that lives in close association with humans, also commonly known as pets, and includes, but is not limited to, canines and felines of any breed. These animals also may include, for example, domesticated farm animals (e.g., cattle, horses, swine, etc.) as well as undomesticated animals held in captivity, e.g., in zoological parks and the like. While foods of any consistency or moisture content are contemplated, in particular the high protein food may be, for example, a wet or dry animal food composition. “Wet” food refers to food which is sold in cans or toil bags and has a moisture content of about 70 to about a 90%. “Dry” food refers to compositions with about 5 to about 15% moisture content and is often manufactured in the form of small bits or kibbles. Also contemplated herein are high protein foods of intermediate moisture consistency and those that may comprise components of various consistency as well as components that may include more than one consistency, for example, soft, chewy meat-like particles as well as kibble having an outer cereal component and an inner cream component,

The protein of the food is obtained from a variety sources such as plants, animals, or both. Animal protein includes meat, meat by-products, dairy, and eggs. Meats include the flesh from poultry, fish, and animals such as cattle, swine, sheep, goats, and the like. Meat by-products include lungs, kidneys, brain, livers, stomachs, and intestines. The protein may also be free amino acids and/or peptides. Preferably, the protein food ingredient comprises meat, a meat by-product, dairy products, or eggs. Meat is meant to include any proteinaceous material obtained from an animal source. Meat includes beef, pork, lamb, fish, chicken, turkey, veal, and the like and mixtures thereof.

The high protein food for use in the present invention can be the final formulation intended for consumption, just the protein component, or the protein component in combination with one or more other components, e.g., fats, carbohydrates, vitamins, and the like. Examples of high protein foods for use in the present invention include animal-derived protein products, e.g., dried egg, poultry meal (e.g., low ash or high ash), chicken meal, lamb meal, pork meal, fish meal, duck meal, venison meal, spray dried chicken, gag chicken, whey protein, and the like.

Sensitive techniques to detect and measure organic acids are known. For example, very small levels of acetic acid can be detected using conventional techniques currently used, for example, to monitor workplace safety. For example, US Occupational Safety and Health Administration (OSHA) Method No. PV2119 provides a system to measure volatile acetic acid levels at 10 ppm (25 mg/m³), wherein samples are collected by drawing a known volume of air through glass sampling tubes containing coconut shell charcoal (SKC Anasorb CSC, lot 2000), then extracted with 0.01 N NaOH and analyzed by ion chromatography (IC) using a conductivity detector. Other systems commercially available from, e.g., Sigma-Aldrich, utilize a capture agent on a solid support, which is then analyzed by gas chromatography (GC). For example, one such system uses a 1-pyrenyldiazomethane (PDAM) derivative on a solid phase microextraction (SPME) fiber as a passive sampling technique for acetic acid vapors. The PDAM-esters are then analyzed by GC. This reaction occurs readily at ambient temperatures without catalysts and the PDAM-ester derivative is stable over a wide temperature and humidity range. Other rapid and accurate techniques of measuring acetic acid in a sample include use of mass spectrometry, e.g., atmospheric pressure chemical ionisation mass spectrometry (APCI-MS) and nuclear magnetic resonance (NMR). Enzyme based assays which measure acetic acid specifically and accurately are also available. Acetic acid (acetate) is converted to acetyl-CoA in the presence of the enzyme acetyl-CoA synthetase (ACS)1, adenosine-5′-triphosphate (ATP) and coenzyme A (CoA). Thus, in one embodiment, acetic acid in a sample is detected in an enzyme-based assay wherein the amount of NADH formed through the combined action of acetyl-CoA synthetase (ACS), citrate synthase (CS) and L-malate dehydrogenase (L-MDH) is measured by increased UV absorption at 340,334 or 365 nm, and the NADH level is used to calculate the acetic acid level. Because of the equilibrium of the indicator reaction, the amount of NADH formed is not linearly (directly) proportional to the acetic acid concentration, but it can nevertheless be reliably calculated. Kits for carrying out such an assay are commercially available, e.g. from NZYTech (Lisbon), Boehringer Mannheim and others.

The invention thus provides, in one embodiment, a method (Method 1) of detecting and/or quantifying food spoilage in a high protein food, comprising:
a) obtaining a sample of the high protein food,
b) determining the level of an acid in the sample, wherein the acid is selected from acetic, propanoic, butanoic, 2-methylbutanoic, 3-methylbutanoic, and 4-methylpentanoic acid, and wherein the level of acid correlates with the degree of food spoilage.
1. 1, Method 1 wherein the acid is acetic acid.
1. 2. Method 1 or 1.1 wherein the level of acid in the sample is detected by means of measuring volatile acid in direct proximity to the sample.
1. 3. Method 1.2 wherein the measurement of volatile acid in direct proximity to the sample is measured by ion chromatography, e.g., by drawing a known volume of air through sampling tubes containing charcoal, then extracting with base, and analyzing by ion chromatography (IC) using a conductivity detector.
1. 4. Method 1.3 wherein the measurement of volatile acid in direct proximity to the sample is measured by gas chromatography, e.g., by drawing a known volume of air through sampling tubes containing a capture agent on a solid support and analyzing by gas chromatography (GC), e.g., by drawing a known volume of air through sampling tubes containing a 1-pyrenyldiazomethane (PDAM) derivative on a solid phase microextraction (SPME) fiber and detecting by GC the PDAM-esters thus formed.
1. 5. Method 1 wherein the level of acid is determined using mass spectrometry, e.g., atmospheric pressure chemical ionisation mass spectrometry (APCI-MS), or using nuclear magnetic resonance (NMR).
1. 6, Method 1, 1.1 or 1.5 wherein the level of acid in the sample is measured by direct analysis of the sample.
1. 7. Method 1.5 wherein the level of acid in the sample is measured by an enzyme assay, e.g., wherein the acid is acetic acid and the amount of NADH formed through the combined action of acetyl-CoA synthetase (ACS), citrate synthase (CS) and L-malate dehydrogenase (L-MDH) is measured by increased UV absorption at 340, 334 or 365 nm, and the amount of NADH is then used to calculate the level of acetic acid in the sample.
1. 8. Method 1.5 or 1.6 wherein the level of acid which indicates spoilage is at least 5000 ppm, e.g., at least 7000 ppm, e.g., at least 10,000 ppm.
1. 9. Any of the foregoing methods wherein a baseline level of acid is established in samples of fresh high protein food, and spoilage is indicated when the level of acetic acid in the test sample of the high protein food is significantly higher, e.g., at least at least one, e.g., at least 2 standard deviations higher, for example 1.5×, 2×, 3×, 4× or 5× higher, than the baseline level.
1. 10. Any of the foregoing methods wherein the high protein food comprises 15-95% crude protein by dry weight.
1. 11. Any of the foregoing methods the high protein food is predominantly, e.g., at least 50%, e.g., at least 90% of animal origin.
1. 12. Any of the foregoing methods the high protein food is a dog or cat food.
1. 13. Any of the foregoing methods wherein the high protein food is an ingredient for a dog or cat food.
1. 14. Any of the foregoing methods wherein the high protein food is or comprises an animal-derived protein product, e.g., selected from dried egg, poultry meal (e.g., low ash or high ash), chicken meal, lamb meal, pork meal, fish meal, duck meal, venison meal, spray dried chicken, gag chicken, whey protein, and combinations thereof.
1. 15. Any of the foregoing methods wherein the high protein food comprises less than 5% fermentable carbohydrate.
1. 16. Any of the foregoing methods wherein the high protein food comprises less than 2% fermentable carbohydrate.
1. 17. Any of the foregoing methods wherein the high protein food comprises meat, meat by-products, dairy, or eggs.
1. 18. Any of the foregoing methods wherein the level of acetic acid correlates with the level of a biogenic amine selected from 1,4-diaminobutane (putrescine), 1,5-diaminopentane (cadaverine), and combinations thereof.
1. 19. Any of the foregoing methods when employed as a quality control measure in the manufacture of a dog or cat food.
1. 20. Any of the foregoing methods wherein the acceptable average levels of acetic acid for a high protein food ingredient include one or more criteria as follows:
   dried egg: <10000 ppm, e.g., <8000 ppm
   poultry meal: <4000 ppm, e.g., <2500 ppm
   lamb meal: <2000 ppm, e.g., <1000 ppm
   pork meal: <2500 ppm, e.g., <2000 ppm
   fish meal: <5000 ppm, e.g., <4000 ppm
   spray dried chicken or gag chicken: <!1000 ppm, e.g., <500 ppm

The invention further provides a method of manufacturing a food comprising a high protein ingredient, e.g., a dog or cat food, wherein spoilage of the high protein ingredient is detected and/or quantified using any of Methods 1, et seq.

The invention further provides a dog or cat a food comprising a high protein ingredient, e.g., a dog or cat food, wherein the high protein ingredient is substantially free of spoilage as detected and/or quantified using any of Methods 1, et seq.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

As used herein and in the appended claims, the singular forms “a”, “an”, and “the” include plural reference unless the context clearly dictates otherwise.

### EXAMPLES

### Example 1

245 samples are analyzed for volatile free fatty acids and biogenic amine levels, the samples consisting of:
107 regular ash poultry meal samples
78 low ash poultry meal samples
17 dried egg samples
14 chicken meal samples
12 lamb meal samples
6 pork meal samples
4 gag chicken samples
4 fish meal samples
1 duck meal sample
1 venison meal sample
1 spray-dried chicken sample

Generally acetic, propanoic and butanoic acid show the highest concentrations in protein-based ingredients, while 2-methylpropanoic, 2-& 3-methylbutanoic, pentanoic, 4-methylpentanoic and hexanoic acid typically have comparatively low levels. However, the actual concentrations differ significantly between individual protein sources:

Dried egg has by far the highest acid content with acetic, propanoic and butanoic acid averaging 8000 ppm, 4500 ppm and 6000 ppm, respectively.

Regular ash poultry meal, low ash poultry meal and chicken meal contain similar acid levels of acetic acid (up to 6500 ppm), propanoic (up to 2000 ppm) and butanoic acid (up to 2500 ppm) but show a high variation of acid concentration between individual lots spanning a range of up to 20-fold.

Fish meal has moderate acid levels. Average acetic acid level concentration is about 4000 ppm, the ones of propanoic and butanoic acid were 500 ppm and 800 ppm, respectively.

Pork meal has slightly lower acid levels with acetic, propanoic and butanoic acid averaging 2000 ppm, 600 and 500 ppm, respectively, followed by lamb meal with approximately 50% lower acid levels.

The lowest acid levels are found in gag chicken and spray-dried chicken.

The level of these acids occurring in the samples is not random. Acetic, propanoic, butanoic, 2- & 3-methylbutanoic and 4-methylpentanoic acid are highly correlated with each other (regression coefficient between 0.8 and 0.95) suggesting a similar formation pathway.

On the other hand, 2-methylpropanoic acid and pentanoic acid show only a weak correlation with the other acids (R²=0.66−0.78) while hexanoic acid does not show any correlation.

### Biogenic Amines:

Within the analyzed sample set, cadaverine and putrescine are the dominating amines in each ingredient while histamine, tyramine, spermine and spermidine are present in significantly lower levels.

Regular and low ash poultry meal has by far the highest acid content with cadaverine and putrescine reaching up to 1600 and 1300 ppm, respectively. These two protein sources also show by far the highest amine variation (up to 50-fold) between lots. Based on current data, this variation is not related to seasonal variation but rather to differences in suppliers and production facilities.

Fish meal, dried egg and duck meal show similar amine biogenic amine concentrations of up to 800 ppm.

Chicken meal contains slightly lower levels of cadaverine and putrescine followed by pork meal with 220 ppm and 180 ppm respectively. All other protein based ingredients show levels below 100 ppm.

The occurring level of these amines is not random. Surprisingly, nearly a linear correlation between cadaverine and putrescine was found, indicating the same route of formation. Further cadaverine and putrescine are loosely correlated with tyramine, indicated by their corresponding regression coefficient of 0.64 and 0.71, respectively. On the other hand, spermine does not show any correlation with another biogenic amine, indicating an independent formation route.

The levels of cadaverine and putrescine correlate with levels of volatile acids. Looking at the correlation of volatile acids and cadaverine in poultry meal, principal component regression shows a reasonable correlation between occurring volatile acids and cadaverine with acetic acid being the main driver, followed by propanoic and butanoic acid with moderate contribution. Since these three acids are also highly correlated with each other, a moderate prediction of occurring cadaverine can be made based solely on the measured acetic acid levels with a correlation coefficient of 0.73.

As expected based on the strong correlation between cadaverine and putrescine, a very similar correlation is found between occurring volatile acids and putrescine. Again, acetic acid is the main driver followed by propanoic and butanoic acid with moderate contribution. Since these three acids are also highly correlated with each other, a moderate prediction of occurring putrescine can be made based solely on the measured acetic acid levels with a correlation coefficient of 0.77.

Based on these correlations, it is possible to predict levels of major biogenic amines based on acetic acid levels. Because these biogenic amine levels correlate with degree of spoilage in the high protein food ingredients tested, spoilage in such foods can be indirectly assessed and quantified based on acetic acid levels.

Using only acetic acid concentrations, the level of cadaverine and putrescine can be predicted with a correlation coefficient of 0.73 and 0.77, respectively. In addition, a weak correlation between acetic acid and tyramine is found as indicated by a 0.49 correlation coefficient. On the other hand, no correlation is found between occurring volatile acids and spermine.

1. A method of detecting and/or quantifying food spoilage in a high protein food, comprising:
a. obtaining a sample of the high protein food, and
b. determining a level of an acid in the sample, wherein the acid is selected from acetic, propanoic, butanoic, 2-methylbutanoic, 3-methylbutanoic, and 4-methylpentanoic acid, and
wherein the level of acid correlates with a degree of food spoilage.

2. The method of claim 1 wherein the acid is acetic acid.

3. The method of claim 1, wherein the level of acid in the sample is detected by means of measuring volatile acid in direct proximity to the sample.

4. The method of claim 1 wherein the level of acid in the sample is measured by direct analysis of the sample.

5. The method of claim 1, wherein a baseline level of the acid is established in samples of fresh high protein food, and food spoilage is indicated when the level of acetic acid in the sample of the high protein food is significantly higher than the baseline level.

6. The method of claim 1, wherein the high protein food comprises 15-95% crude protein by dry weight.

7. The method of claim 1, wherein the high protein food is an ingredient for a dog or cat food.

8. The method of claim 1, wherein the high protein food is or comprises an animal-derived protein product selected from dried egg, poultry meal (e.g., low ash or high ash), chicken meal, lamb meal, pork meal, fish meal, duck meal, venison meal, spray dried chicken, gag chicken, whey protein, and combinations thereof.

9. The method of claim 1, wherein the high protein food comprises less than 5% fermentable carbohydrate.

10. The method of claim 1, wherein the level of the acid correlates with a level of a biogenic amine selected from 1,4-diaminobutane (putrescine), 1,5-diaminopentane (cadaverine), and combinations thereof.

11. A method for manufacturing a dog or cat food, comprising utilizing the method of claim 1 as a quality control measure.

12. A method of manufacturing a food comprising a high protein ingredient, wherein spoilage of the high protein ingredient is detected and/or quantified using the method of claim 1.

13. A dog or cat food comprising a high protein ingredient, wherein the high protein ingredient is substantially free of spoilage as detected and/or quantified using the method of claim 1.
